(19) United States
(12) Patent Application Publication
(10) Pub. No.: US 2026/0326041 A1
(43) Pub. Date: Oct. 22, 2026

(54) CAPPING INITIATED AND PRESSURE REGULATED PLASMA SEPARATION DEVICE

(71) Applicant: ZYMERON CORPORATION, Research Triangle Park, NC (US)

(72) Inventors: Meng SUN, Wake Forest, NC (US); Zhiguo ZHOU, Chapel Hill, NC (US); Haidong FENG, Durham, NC (US)

(21) Appl. No.: 19/535,834

(22) Filed: Feb. 10, 2026

Related U.S. Application Data
(60) Provisional application No. 63/757,513, filed on Feb. 12, 2025.

(51) Int. Cl. B01D 69/02; A61B 5/15; B01D 63/08; B01L 3/00

(52) U.S. Cl. CPC B01D 69/02; A61B 5/150755; B01D 63/082; B01L 3/502; B01D 2313/10; B01D 2313/12; B01D 2313/18; B01D 2325/022; B01D 2325/02833; B01D 2325/02834; B01L 2200/026; B01L 2200/0631; B01L 2300/042; B01L 2300/047; B01L 2300/0681; B01L 2300/0832; B01L 2300/14; B01L 2300/161; B01L 2400/0406; B01L 2400/0487; B01L 2400/0688; B01L 2400/086; B01L 2400/088

(57) ABSTRACT

Described herein is plasma separation device, which does not require any reagents or other apparata, that processes a microliter to milliliter-scale volume of blood and separates plasma through application of an active positive pressure upon a manual assembling activation of the device. Also described are methods for separating plasma from blood using the device.

### CROSS-REFERENCE TO RELATED APPLICATIONS

This application claims priority to U.S. Provisional Patent Application No. 63/757,513, filed on Feb. 12, 2025, which is incorporated by reference herein in its entirety.

### BACKGROUND

The separation of blood into its cellular and plasma components is a critical step in numerous medical applications, including diagnostic testing. Isolated plasma is particularly valuable in rapid diagnostics due to its ability to provide a clear, cell-free sample for the detection of biomarkers, pathogens, or other analytes of clinical interest. The use of plasma in diagnostics enables high sensitivity and specificity in assays, as it avoids interference from blood cells or other particulate matter present in whole blood.

Traditional methods for plasma isolation, such as centrifugation, are often time-consuming, require bulky equipment and electric power; thus, these methods are not friendly for point-of-care or decentralized tests. In contrast, membrane-based separation methods offer several advantages including: (1) speed: membrane-based separation methods can isolate plasma rapidly to significantly reduce the processing time compared to centrifugation. (2) compactness and portability: membrane-based plasma separation devices are typically compact, lightweight, and do not require electricity to operate; (3) cost-effectiveness: the relatively simple design of such devices reduces manufacturing and operational costs. Therefore, membrane-based plasma separation can lower the cost per test thereby enabling more affordable diagnostic solutions for broad diagnostic applications that demand rapid point-of-care, bedside, or remote testing results.

Membrane-based plasma separation presents engineering challenges across device design, surface chemistry, and materials science, as the separator configuration must be carefully tuned to achieve reliable performance. Common failure modes include leakage, hemolysis, sample carryover/contamination, and low plasma yield. In practice, separation success is governed by the coupled effects of fluidic control, surface wettability, membrane properties (e.g., pore size and structure), and device geometry. As a result, many membrane-based systems require auxiliary components or reagents—such as pumps or vacuum sources, magnetic beads, or chemical additives—to initiate, accelerate, or complete plasma separation

What is needed is a capping initiated plasma separation device that does not require any reagents or other apparata and method for use thereof.

### SUMMARY

One embodiment described herein is a fluid separation device comprising: a sample collection receptacle for receiving a fluid sample, the sample collection receptacle including: an inlet for receiving the fluid sample, a separation chamber in fluid communication with the inlet, the separation chamber having a membrane to filter the fluid sample, an outlet in fluid communication with the separation chamber, and at least one hydrophilic guide member coupled to the outlet; and a cap selectively coupled to the sample collection receptacle, the cap including a pressure chamber in fluid communication with the inlet; wherein, when the cap is coupled to the sample collection receptacle, the pressure chamber exerts a compression force through the inlet to move the fluid sample through the membrane and into the outlet.

Another embodiment described herein is a fluid separation device comprising: a sample collection receptacle for receiving a fluid sample, the sample collection receptacle including: an inlet for receiving the fluid sample, a separation chamber in fluid communication with the inlet, the separation chamber having a membrane to filter the fluid sample, a fluid distributor configured to distribute the fluid sample across the membrane, wherein the fluid distributor comprises, an inner surface of the separation chamber, a manifold, or an insert, an outlet in fluid communication with the separation chamber, and at least one hydrophilic guide member coupled to the outlet; and a cap selectively coupled to the sample collection receptacle, the cap including a pressure chamber in fluid communication with the inlet; wherein, when the cap is coupled to the sample collection receptacle, the pressure chamber exerts a compression force through the inlet to move the fluid sample through the membrane and into the outlet. In one aspect, the fluid separation device further comprises: a flange partially forming the outlet of the sample collection receptacle; and a collection vessel removably coupled to the flange. In another aspect, the fluid separation device further comprises: an interface partially forming the outlet of the sample collection receptacle and facilitating fluid communication with an analytical device. In another aspect, the fluid separation device further comprises a fluid regulator for selectively permitting fluid communication between the inlet and the separation chamber. In another aspect, the fluid regulator is a capillary valve, a layer of mesh with pore size of 50 to 200 μm, or a thin film configured to rupture under an applied load. In another aspect, the fluid separation device further comprises a support disposed at a lower portion of the membrane, the support being configured to support the membrane, wherein the support facilitates fluid communication between the separation medium and the outlet of the sample collection receptacle. In another aspect, the inlet is configured to contain a fixed volume of the fluid sample. In another aspect, when a force is applied to the device, the fluid distributor permits fluid communication between the inlet and the separation chamber by releasing the fixed volume of the fluid sample. In another aspect, the at least one hydrophilic guide member extends from the outlet of the sample collection receptacle substantially vertically in a direction of the compression force. In another aspect, the at least one hydrophilic guide member extends from the outlet of the sample collection receptacle at an angle of at least 10° from a horizontal plane perpendicular to a direction of the compression force. In another aspect, the at least one hydrophilic guide member comprises a hydrophilic material, the hydrophilic material comprising a liquid drop contact angle θ<65°. In another aspect, the inlet has a liquid drop contact angle θ<90°. In another aspect, the membrane has an average porosity of no less than 40%. In another aspect, the membrane at least has a minimum pore size of 0.1 to 2 μm.

Another embodiment described herein is a fluid separation device, comprising: a sample collection receptacle for receiving a fluid sample, the sample collection receptacle including: an inlet for receiving the fluid sample; a separation chamber in fluid communication with the inlet, the separation chamber having a membrane to filter the fluid sample; a fluid regulator for selectively permitting fluid communication between the inlet and the separation chamber; a fluid distributor to distribute the fluid sample across the membrane; an outlet in fluid communication with the separation chamber; and at least one hydrophilic guide member coupled to the outlet; and a cap selectively coupled to the sample collection receptacle, the cap including a pressure chamber in fluid communication with the inlet; wherein, when the cap is coupled to the sample collection receptacle, the pressure chamber exerts a compression force through the inlet to move the fluid sample through the membrane and into the outlet.

Another embodiment described herein is a method for separating a fluid sample comprising: applying a fluid sample to the fluid separation device described herein; exerting a force onto the device to permit a volume of the fluid sample to pass through a fluid regulator into the separation chamber; coupling the cap to the sample collection receptacle to exert a compression force; separating the fluid sample in the membrane of the separation chamber; and collecting a separated aliquot from the fluid sample via the outlet in fluid communication with the separation chamber. In one aspect, the fluid sample is a blood sample. In another aspect, the separated aliquot is plasma. In another aspect, the method further comprises: affixing a collection vessel to a flange of the outlet on the fluid separation device. In another aspect, the separated aliquot is collected in about 30-180 seconds.

Another embodiment described herein is a kit comprising: one or more the fluid separation devices described herein; a means for obtaining a blood sample from a human or animal subject; optionally, a blood collection vial; optionally, one or more antiseptics; optionally one or more pairs of gloves; and packaging, labels, and instructions for use.

Another embodiment described herein is the use of the fluid separation device described herein to separate plasma from a blood sample.

### DESCRIPTION OF THE DRAWINGS

The drawings below illustrate embodiments for the design, principle, mechanism, and operation of the disclosed plasma separation device. The drawings are not drawn to scale and are intended to illustrate the components and functions of the device.

FIG. 1 shows a zoomed-out, perspective view of a plasma separation device.

FIG. 2 shows a side, cross-sectional view of the plasma separation device of FIG. 1 in a pre-separation stage of the device.

FIG. 3 shows a side, cross-sectional view of the plasma separation device in a separation stage of the device.

FIG. 4A shows an exploded cross-sectional view of the plasma separation device in the post-separation stage of the device.

FIG. 4B shows a side, cross-sectional view of the plasma separation device in the separation stage of the device.

FIG. 5 shows a detailed, cross-sectional view of the plasma separation device of FIG. 1.

FIG. 6A-D illustrate a process of using the plasma separation device of FIG. 5 including the steps of sampling blood in (FIG. 6A); wetting the separation medium (FIG. 6B); capping the device for plasma separation (FIG. 6D); and plasma collection (FIG. 6D).

FIG. 7 shows a perspective view of a sample collection receptacle for use with the plasma separation device of FIG. 5.

FIG. 8 shows a detailed, cross-sectional view of a plasma separation device according to another embodiment of the disclosure.

FIG. 9 shows a side, cross-sectional view an alternative embodiment of a cap for use with the plasma separation device of FIG. 5

FIG. 10A shows the plasma on the collection film hydrophilic guide member.

FIG. 10B shows the plasma in a collection tube.

FIG. 10C shows an image of a hemacytometer showing cleared plasma without red and white blood cells.

FIG. 11 shows a bar graph showing the concentration of hemoglobin collected with hematocrit (HCT) levels of 35%, 42%, and 48%.

FIG. 12 shows a bar graph showing the amount of CMV IgG collected with the apparatus disclosed herein (referred to as “RapiSep”) as compared to plasma collected with centrifugation.

FIG. 13 shows examples of fluid regulators, from left to right an inlet without a regulator, with a valve, a mesh, and a film.

FIG. 14A-C show examples of the hydrophilic guide member. FIG. 14A shows an outlet without a guide. FIG. 14B shows an outlet with a film. FIG. 14C shows an outlet with a tube.

FIG. 15A-C show examples of a fluid distributor. FIG. 15A shows a fluid distributor including a curved surface. FIG. 15B shows fluid distributor including a manifold. FIG. 15C shows a fluid distributor including an insert with a porous membrane.

FIG. 16A-F show detailed perspective views of different embodiments of fluid distributors including a side, perspective view of a fluid distributor including a flat surface (FIG. 16A), a cross-sectional view of the fluid distributor of FIG. 16B, a side-cross-sectional view of a fluid distributor including a curved surface (FIG. 16C), a side cross-sectional view of a fluid distributor including a surface with ridges (FIG. 16D), a side cross-sectional view of a fluid distributor including a manifold with vertical ports (FIG. 16E), and a side cross-sectional view of a fluid distributor including a manifold with horizontal ports (FIG. 16F).

FIG. 17 shows a perspective view of a medium support that supports the separation medium and contributes to the collection of plasma in the outlet.

### DETAILED DESCRIPTION

Unless otherwise defined, all technical and scientific terms used herein have the same meaning as commonly understood by one of ordinary skill in the art. For example, any nomenclatures used in connection with, and techniques of medicine, clinical chemistry, biochemistry, molecular biology, immunology, microbiology, genetics, and cell and tissue culture described herein are well known and commonly used in the art. In case of conflict, the present disclosure, including definitions, will control the interpretation. Exemplary methods and materials are described below, although methods and materials similar or equivalent to those described herein can be used in practice or testing of the embodiments and aspects described herein.

As used herein, terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not. As used herein, “comprising,” is an “open-ended” term that does not exclude additional, unrecited elements or method steps. As used herein, “consisting essentially of” limits the scope of a claim to the specified materials or steps and those that do not materially affect the basic and novel characteristics of the claimed invention. As used herein, “consisting of” excludes any element, step, or ingredient not specified in the claim.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “~” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points, or as described above in the definition of “about.”

As used herein, the terms “room temperature,” “RT,” or “ambient temperature” refer to the typical temperature in an indoor laboratory setting. In one aspect, the laboratory setting is climate controlled to maintain the temperature at a substantially uniform temperature or with a specific range of temperatures. In one aspect, “room temperature” refers a temperature of about 15-30 °C, including all integers and endpoints within the specified range. In another aspect, “room temperature” refers a temperature of about 15-30 °C; about 20-30 °C; about 22-30 °C; about 25-30 °C; about 27-30 °C; about 15-22 °C; about 15-25 °C; about 15-27 °C; about 20-22 °C; about 20-25 °C; about 20-27 °C; about 22-25 °C; about 22-27 °C; about 25-27 °C; about 15 °C±10%; about 20 °C±10%; about 22 °C±10%; about 25 °C±10%; about 27 °C±10%; ~20 °C, ~22 °C, ~25 °C, or ~27 °C, at standard atmospheric pressure.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the terms “separation medium” and “membrane” may be used interchangeably. Separation medium can represent the single layer membrane or membrane system comprising multiple membranes or discrete membranes.

Described herein is a separation device comprising a sample collection receptacle for receiving a fluid sample and a cap selectively coupled to the sample collection receptable.

The sample collection receptacle for receiving the fluid sample may comprise an inlet for receiving the fluid sample. The inlet may comprise any material that allows for the inlet to have a liquid drop contact angle (θ) less than or equal to 90°. In some embodiments, the liquid drop contact angle (θ) may be less than 90°. In some embodiments, the liquid drop contact angle of the material comprising the inlet may be less than 85°, less than 80°, less than 75°, less than 70°, less than 65°, less than 60°, less than 55°, or less than 50°.

In some embodiments, the inlet includes at least one opening that allows for the sample to be added to the inlet. The sample may be added to the inlet by any means known to a person of ordinary skill in the art. In some embodiments, the inlet may be configured to contain a fixed volume of a fluid sample. In some embodiments, the inlet may hold about 10-1000 μL of a fluid sample. In some embodiments, the inlet may have a cross-section of 0.5 to 5 mm. In some embodiments, the inlet may have a cross-section of 0.5 mm, 0.6 mm, 0.7 mm, 0.8 mm, 0.9 mm, 1 mm, 1.5 mm, 2 mm, 2.5 mm, 3 mm, 3.5 mm, 4 mm, 4.5 mm, 5 mm, or greater. In some embodiments, the inlet may have a cross-section of 0.5-1 mm, 0.5-1.5 mm, 0.5-2 mm, 0.5-2.5 mm, 0.5-3 mm, 0.5-3.5 mm, 0.5-4 mm, 0.5-4.5 mm, 0.5-5 mm, 1-2 mm, 1-2.5 mm, 1-3 mm, 1-3.5 mm, 1-4 mm, 1-4.5 mm, 1-5 mm, 2-3 mm, 2-3.5 mm, 2-4.5 mm, 2-5 mm, 3-4 mm, 3-4.5 mm, 3-5 mm, or 4-5 mm, including all endpoints, integers, decimals, and subranges within the range.

The sample collection receptacle may also comprise a separation chamber. In some embodiment, the separation chamber may be in fluid communication with the inlet. In some embodiments, the separation chamber may comprise a membrane to filter a fluid sample. The membrane may be any material having a microporous structure that can retain particles. In some embodiments, the membrane may comprise one-, two-, three-, four-, five, or more layers of material. Exemplary membrane materials having a microporous structure include, but are not limited to, uniform pore filter papers, glass microfiber-based retention membranes, and asymmetric pore-based separation membranes. In some embodiments, the membrane may comprise a plurality of layers comprising cellulose, polycarbonate (PCTE), polyethersulfone (PES), polyester (PETE), polyethylene (PE), and polypropylene (PP), or a combination thereof.

In preferred embodiments, the membrane may be an asymmetric membrane pore-based separation membrane. As used herein, an “asymmetric membrane” comprises pores with varying diameters or porosity. In one aspect, an asymmetric membrane comprises pores of 40-50 μm, including all integers, fractions, and subranges within the specified range on the blood loading side, and pores of 1-2 μm, including all integers, fractions, and subranges within the specified range on the underside of the membrane (opposite the blood loading side). Asymmetric membranes can be made by controlled etching, adjustment of liquid vesicle composition, and electrospinning.

In some embodiments, the membrane comprises pores with a maximum size of 10-100 μm, including all integers, fractions, and subranges within the specified range, and a minimum size of 0.1-2 μm, including all integers, fractions, and subranges within the specified range. In some embodiments, the membrane may have an average pore size of 0.1-100 μm including all integers, fractions, and subranges within the specified range. In some embodiments, the membrane comprises a maximum pore size of 10, 20, 30, 40, 50, 60, 70, 80, 90, or 100 μm. In some embodiments, the membrane comprises a maximum pore size of 10-20, 20-30, 30-40, 40-50, 50-60, 60-70, 70-80, 80-90, or 90-100 μm. In some embodiments, the membrane comprises a minimum pore size of 0.1, 0.2, 0.3. 0.4, 0.5, 0.6, 0.7, 0.8, 0.9, 1.0, 1.1, 1.2, 1.3, 1.4, 1.5, 1.6, 1.7, 1.8, 1.9, or 2.0 μm. In some embodiments, the membrane comprises a minimum pore size of 0.1-0.4, 0.4-0.6, 0.6-0.8, 0.8-1.0, 1.0-1.2, 1.2-1.4, 1.4-1.6, 1.6-1.8, 1.8-2.0 μm. In some embodiments, the membrane has a pore size below the size of a blood cell (~6.2-8.2 μm) so that blood cells do not pass through the membrane.

In some embodiments, the membrane comprises a pore size of 50 to 200 nanometers (nm). In some the membrane may have an average pore size of about 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, or greater. In some embodiments, the membrane may have an average pore size of about 50-75 nm, 50-100 nm, 50-125 nm, 50-150 nm, 60-75 nm, 60-100 nm, 60-125 nm, 60-150 nm, 70-100 nm, 70-125 nm, 70-150 nm, 80-100 nm, 80-125 nm, 80-150 nm, 90-125 nm, 90-150 nm, 100-125 nm, 100-150 nm, 110-125 nm, 110-150 nm, 120-150 nm, 130-150 nm, or 140-150 nm, including all endpoints, integers, fractions, and subranges within the specified ranges. In some embodiments, the membrane may have an average pore size of no less than 200 nm, no less than 175 nm, no less than 150 nm, no less than 125 nm, no less than 100 nm, no less than 75 nm, or no less than 50 nm. In some embodiments, the membrane may have an average pore size of no greater than 50 nm, no greater than 75 nm, no greater than 100 nm, no greater than 125 nm, no greater than 150 nm, no greater than 175 nm, or no greater than 200 nm. In some embodiments, the membrane has an average pore size below the size of a blood cell (~6.2-8.2 μm) so that blood cells do not pass through the membrane.

In some embodiments, the membrane comprises a porosity of 40% to 90%. In some the membrane may have an average porosity of about 40%, 45%, 50%, 55%, 60%, 65%, 79%, 75%, 80%, 85%, 90%, 95%, or even greater. In some embodiments, the membrane may have an average porosity of about 40-50%, 40-60%, 40-70%, 40-80%, 40-90%, 50-60%, 50-70%, 50-80%, 50-90%, 60-70%, 60-80%, 60-90%, 70-80%, 70-90%, 80-90%, or 90-95%, including all endpoints, integers, fractions, and subranges within the specified ranges. In some embodiments, the membrane may have an average porosity of no less than 40%, no less than 50%, no less than 60%, no less than 70%, no less than 80%, or no less than 90%. In some embodiments, the membrane may have an average porosity of no less than 40-90%. In some embodiments, the membrane may have an average porosity of no greater than 40%, no greater than 50%, no greater than 60%, no greater than 70%, no greater than 80%, or no greater than 90%. In some embodiments, the membrane may have an average porosity of no greater than 40-90%. In some embodiments, the membrane has an average porosity of 80%, balanced between blood cell retention capability and plasma flow velocity so that the blood cells do not pass through the membrane while the plasma can rapidly pass through.

In some embodiments, the membrane may have various shapes such as circles, squares, triangles, trapezoids, parallelograms, etc. In some embodiments, the membrane may have various cross-sectional thicknesses. The membrane layer may be in a continuous form or a composite of discrete membrane sections, where it is divided into individual pieces within a single layer. The separation capability is associated with the total surface area of membrane for a single layer membrane or a plurality of layers of membranes.

In some embodiments, the surface area of the membrane ranges from 0.2 to 50 cm².

In some embodiments, the size of the membrane may correspond with the amount of pressure that is applied by the cap. In some embodiments, the dimensions of the membrane ranges from 4-60 mm. The size of the membrane may be tunable for the amount of fluid that a user desires to separate.

The sample collection receptacle may also comprise a fluid regulator. The fluid regulator may selectively permit fluid communication between the inlet and the separation chamber. In some embodiments, a force may be applied to the fluid regulator to permit the fluid sample to pass through the fluid regulator into the sample chamber. The fluid regulator can be configured to retain the input volume of blood without requiring an additional metering tool such as a pipette.

In some embodiments, the fluid regulator may be any functional component such as a valve, a mesh, or a thin film. Exemplary mesh materials include, but are not limited to PE, PETE, PTFE, PES. To be functional as described, the pore size of the mesh should be in the range of 50-200 μm. Exemplary thin film materials that may be used include, but are not limited to plastic, polytetrafluoroethylene (PTFE), polymeric films, mylar, aluminum or other metal foils, or the like. In some embodiments, the inner surface of the fluid regulator that makes contact with the sample should not absorb or adsorb any of the sample or components thereof.

The sample collection receptacle may also comprise a fluid distributor in fluid communication with the inlet and the separation chamber. The fluid distributor may be configured to guide a flow direction of the fluid sample and promote substantially uniform distribution of the fluid sample across the surface of the membrane. In some embodiments, the fluid distributor may comprise a surface of the separation chamber adjacent to the inlet. The surface may be flat, curved, or include one or more ridges. In some embodiments, the surface may contact the membrane. In some embodiments, the surface may be spaced apart from the membrane and configured to direct the fluid sample from the inlet across the membrane.

In some embodiments, the fluid distributor may comprise a manifold in fluid communication with the inlet and the separation chamber, the manifold configured to divide the fluid sample from the inlet into a plurality of channels prior to contacting the membrane.

In some embodiments, the fluid distributor may comprise an insert positioned within the separation chamber between the inlet and the membrane. The insert may be composed of a hydrophilic film, porous membrane, mesh, or combinations thereof, and may be configured to promote fluid flow via capillary action.

The sample collection receptacle may also comprise an outlet in fluid communication with the separation chamber. In some embodiments, the outlet is composed of a hydrophilic material. Exemplary materials that may be used to form the outlet include, but are not limited to, plastic, derivatized or coated plastics, paper, or glass. In some embodiments, a flange on the sample collection receptacle may partially form the outlet of the sample collection receptacle. In some embodiments, the outlet can include an interface that partially forms the outlet of the sample collection receptable to facilitate fluid communication with an analytical device. The analytical device may detect one or more analytical measure. The analytical measurement may include, but is not limited to, the presence of target analyte, content of separation aliquot, volume of the separation aliquot, or the purity of separation aliquot.

The sample collection receptacle may also comprise at least one hydrophilic guide member coupled to the outlet. In some embodiments, the hydrophilic guide member may be configured to facilitate movement of the fluid sample from the outlet. The hydrophilic guide membrane may extend from the outlet in any manner or direction that permits the movement of the separated fluid. In some embodiments, the hydrophilic guide member may extend from the outlet of the sample collection receptacle substantially vertically in the direction of a force applied to facilitate the movement of the sample through the sample collection receptacle. In some embodiments, the hydrophilic guide member may extend from the outlet of the sample collection receptacle at an angle of at least 10° from a horizontal plane perpendicular to the direction of a force applied to facilitate movement of the sample through the sample collection receptacle.

In some embodiments, the hydrophilic guide member may be any material having a liquid drop contact angle (θ) of less than 90°. In some embodiments, the hydrophilic guide member may be any material having a liquid drop contact angle (θ) of less than 65°. Exemplary materials that may be used for the hydrophilic guide member include, but are not limited to, plastic, derivatized or coated plastics, paper, glass, or combinations thereof.

In some embodiments, the hydrophilic guide member may be a thin film or a tube, which facilitates the plasma being drawn out from the receptacle to a collection vial. In some embodiments, the thin film leaves no plasma residue as compared to the tube option, which may leave plasma residue on the inside of the tube and trap a portion of plasma volume inside the tube.

The sample collection receptacle may also comprise a medium support disposed at a lower portion, or bottom of the separation chamber to support the separation medium. This medium support elevates the separation medium from the bottom surface and provides flow paths for the plasma, guiding the flow to the outlet of the sample collection receptacle.

The sample collection receptacle may also comprise a collection vessel. In some embodiments, the collection vessel is removably coupled to the flange on the outlet.

The cap of the fluid separation device may further comprise a pressure chamber in fluid communication with the inlet of the sample collection receptacle. In some embodiments, when the cap is coupled to the sample collection receptacle the pressure chamber exerts a compression force through the inlet to induce the movement of the fluid sample through the membrane and into the outlet.

The cap may optionally comprise a plunger. In some embodiments, a user can depress the plunger to exert a compression force through the inlet and induce the movement of the fluid sample through the membrane and into the outlet.

In some embodiments, the fluid separation device is a plasma separation apparatus that is hand-held and self-contained for plasma separation from whole or diluted blood. The device processes tens of microliters of blood, producing clear, contamination-free plasma at a yield of at least 20% of the input blood volume, which makes is advantageous for rapid diagnostics. The device is not limited to rapid diagnostics and may be applied to other applications.

The fluid separation device disclosed herein may separate plasma from blood quickly by addressing the potential issues associated with slower plasma separation techniques or those that require additional reagents or apparata such as pumps or centrifuges. Other techniques involve using centrifugation or passive filtration/sedimentation methods and can take 5-10 minutes to complete a separation. The fluid separation device disclosed herein is a stand alone apparatus that does not require any reagents or other apparata and can be used to separate plasma in seconds (less than 1 min).

The fluid separation device disclosed herein provides precise and active control on plasma separation through a manual activation mechanism that applies a regulated, constant pressure to the separation. The separation may be performed with minimal effort as a result of a unique, engineered design of the plasma separation device, particularly at the interface of a sealing cap and a sample collection receptacle. As the cap and sample collection receptacle are assembled, an air-tight chamber with reduced internal volume and air compression creates a positive pressure. Consequently, plasma separation occurs almost instantaneously upon assembly of the device without any time delay, or any other operation steps involved. The entire process takes less than 15 seconds from start to finish, offering a rapid and reliable method for plasma separation while maintaining consistent separation performance and high plasma quality. In some embodiments, a fluid regulator may exist in the fluid separation device to control or limit the volume of the sample and the plasma separation may occur after the fluid regulator is released and permits the flow of blood from the inlet to the membrane in the separation chamber.

The fluid separation device disclosed herein was designed to allow any untrained non-professional users to operate it by following simple steps provided in manual instructions. Manipulations of the device include: (1) collect blood sample, (2) assemble fluid separation device (3) activate separation by: (i) contacting the sample to the device, (ii) tapping, and (iii) pushing and pulling lightly with minimum effort on using this device, and (4) collecting plasma.

The fluid separation device disclosed herein is distinguished from other apparata where the separated plasma is absorbed on paper-based substrates because the fluid separation device is configured to collect free plasma in liquid form. The yield is between 20-40% of the total input blood volume and the separation efficiency is 50-70% depending on the hematocrit of the blood. In some embodiments, the fluid separation device disclosed herein may use only one or two drops of blood, e.g., 35 μL in volume, and will yield 12 μL plasma. In some embodiments, the fluid separation device disclosed herein may use sub-milliliter blood in volume, and will yield 0.2 mL plasma. With the fluid separation device disclosed herein this volume may be sufficient for most downstream diagnostic applications.

The fluid separation device disclosed herein possesses rapid performance including but not limited to separation time, plasma yield, and quality in the matter of clearness and contamination level by blood cells and hemolysis.

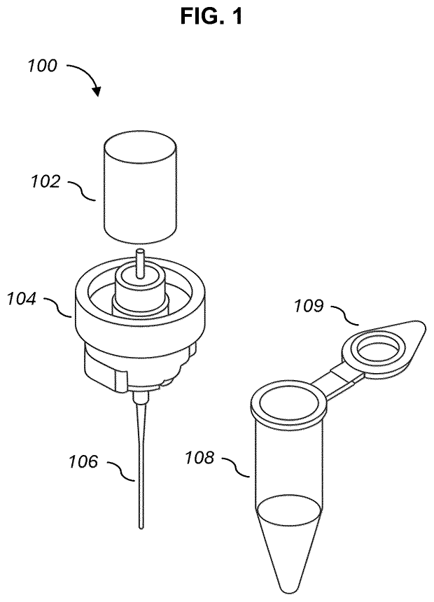

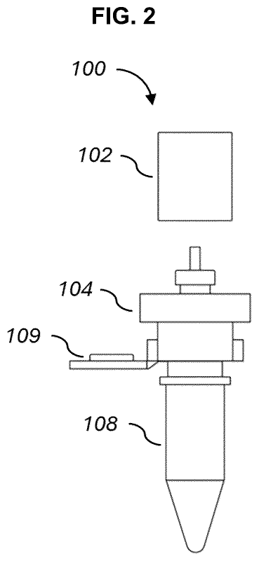

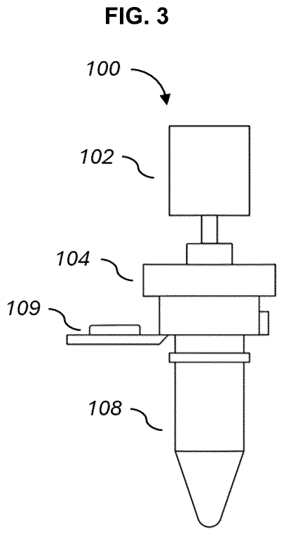

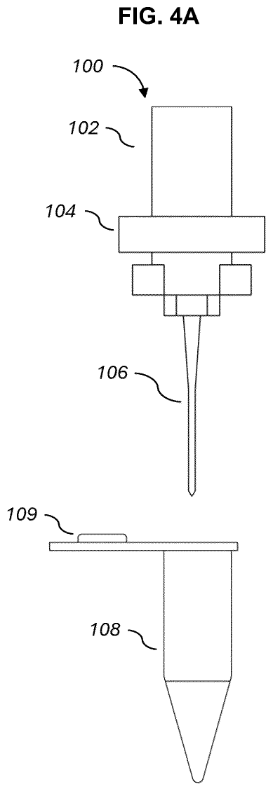

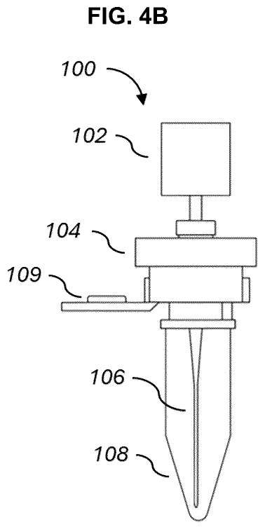

FIG. 1-5 illustrate a fluid separation device 100 including a sealing cap 102, a sample collection receptacle 104, and a collection vial 108. In some embodiments, the fluid separation device 100 is a blood and plasma separation device for separating plasma from whole blood. In the illustrated embodiment, the collection vial 108 also includes a cover 109 for selectively sealing the vial 108.

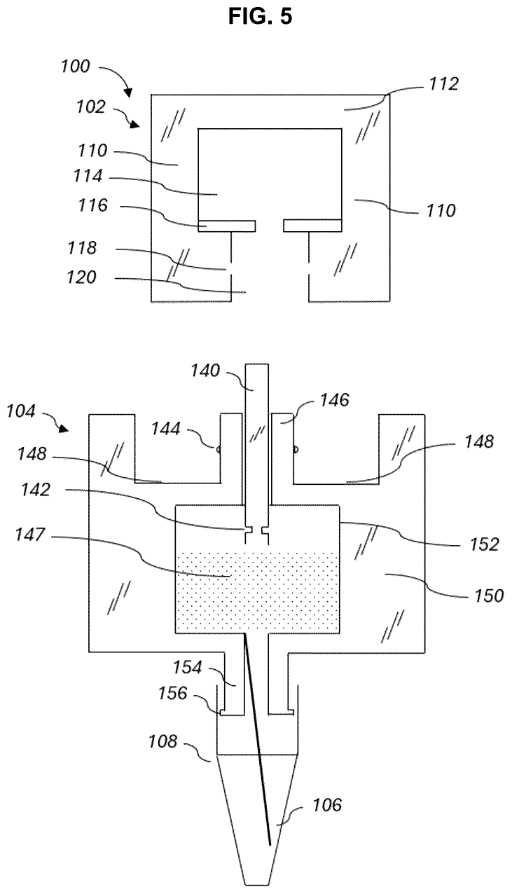

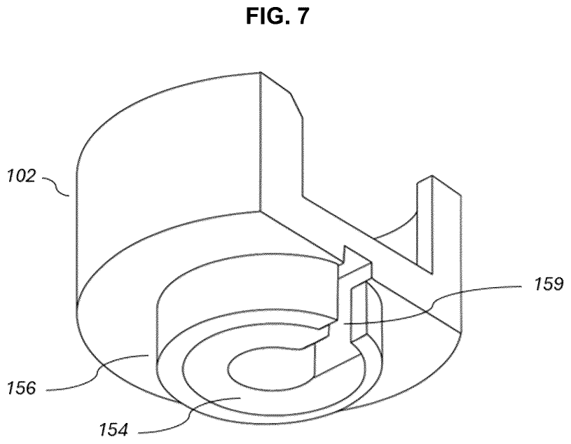

With reference to FIG. 5, the sample collection receptacle 104 defines a main body 150 for housing a plasma separation chamber 152 having a plasma separation medium 147 disposed therewithin, a blood inlet 140 supported on the main body 150 and in selective fluid communication with the plasma separation chamber 152, and a plasma outlet 154 in fluid communication with the plasma separation chamber 152 and located downstream from the plasma separation chamber 152. The plasma outlet 154 includes a vent 159 (FIG. 7) configured to expose the plasma outlet 154 to ambient air pressure to facilitate resistance-free plasma flow into the collection vial 108. The blood inlet 140 is supported on a post 146 extending from the main body 150 and is located upstream from the plasma separation chamber 152. The blood inlet 140 includes a fluid regulator 142 configured to control or regulate aspects of fluid within the inlet 140, such as fluid flow, etc. The illustrated fluid regulator 142 is a valve, such as a capillary valve, configured to selectively allow a fixed volume of a whole blood sample to enter the plasma separation chamber 152. The illustrated valve 142 functions to selectively prevent the flow of blood through the valve 142 and retain the fixed volume of blood within the blood inlet 140 until the blood level reaches a predetermined capacity. In some embodiments, the valve 142 retains the fixed volume of blood until the user breaks the valve 142 permitting the fixed volume of blood to flow into the plasma separation chamber 152, which is explained in greater detail below. In other embodiments, the valve 142 allows passive diffusion of the fixed volume of blood through the valve 142 and into the plasma separation chamber 152 by capillary action in response to gravitational force. In yet other embodiments, the blood inlet 140 may include a fill line that indicates the required fixed volume of the blood sample has been successfully collected. In yet other embodiments, the fluid regulator 142 can be any other conventional type of functional component known in the art.

In some embodiments, a cross section of the blood inlet 140 can be square, circular, or rectangular in shape and the fixed volume of the blood sample may be controlled by the internal volume of the blood inlet 140 that follows the equation below:

V=L×A.

For example, a cylindrical shaped blood inlet 140 can be modeled using the equation below:

V=L×πr²,

where A is the area of the cross section, and L and r are the length and the radius of the blood inlet 140, respectively. Next, a strong capillary pressure, P, for the selected inlet geometry of the blood inlet 140 can be modeled according to the Young-Laplace equation:

$$P = \frac{2 γ cos θ}{r} ,$$

where γ is free energy of the liquid-vapor interface, and e is the contact angle. This calculated capillary pressure value ensures that the fluid regulator 142 allows for adequate pressure for driving spontaneous blood flow through the fluid regulator 142. In other embodiments, the blood inlet 140 is hydrophilic (θ<90°), and the diameter or the size of the cross section of the blood inlet 140 is about 1-2 mm.

With continued reference to FIG. 5, the plasma separation medium 147 includes a blood separation membrane layer for isolating the blood cells from the plasma of the whole blood sample. In some embodiments, the plasma separation medium 146 can be circular, rectangular, trapezoidal, elliptical, or any other type of polygon. In other embodiments, the plasma separation medium 146 may have dimensions that are constrained by the plasma separation chamber 152 in the sample collection receptacle 104, with dimension ranging from 4-25 mm. This range is determined by the plasma separation medium 146 separation capability, and the total separation time of the fluid sample. The separation membrane layer can be connected or adhered to the sample collection receptacle 104 by any means known to a person of ordinary skill in the art. In some embodiments, the separation membrane layer may be connected and sealed inside the sample collection receptacle 104 utilizing a bonding method, such as a pressure sensitive adhesive, thermal bonding, pressure fitting, or liquid gaskets. In other embodiments, a gap may exist between the blood inlet 140 and plasma separation medium 147, with a gap distance ranging from 0.05 mm to 20 mm. The gap assists the blood dispersion in the plasma separation medium 146 and regulates pressure distribution inside the plasma separation chamber 152. The gap distance can be controlled by methods including, but not limited to, a geometry control and spacer ring. In other embodiments, an additional supportive layer (not shown) is disposed under the plasma separation medium 146 to provide additional support to the plasma separation medium 146 and regulate plasma flow.

In some embodiments, a single layer or a plurality of layers of blood separation membranes may be used and implemented in the plasma separation medium 147. In some embodiments, the plasma separation medium 146 can include a plurality of membrane disc filters that form a multiple layer membrane system as a bottom layer to prevent blood cell leakage into the plasma. In some embodiments, the plurality of pore filters may be made of cellulose, polycarbonate (PCTE), polyethersulfone (PES), polyester (PETE), polyethylene (PE), or polypropylene (PP), and have a pore size smaller than the conventional size of blood cells.

In other embodiments, the glass fiber-based membrane layer can be configured as a multiple membrane layer system, in which additional separation membranes are set underneath the glass fiber membrane and contact the plasma outlet 154. This additional membrane provides sufficient capillary pressure to absorb blood plasma from the glass fiber membrane and prevent blood cells from entering the separated plasma.

In some embodiments where the asymmetric membrane is the blood separation membrane layer, the asymmetric membrane includes a pore size ranging from 50 μm to 0.2 μm at different depths of the membrane. By varying the pore size, the larger holes near the blood inlet 140 provide sufficient space for blood cell trapping while simultaneously preventing membrane fouling. In contrast, smaller holes near the plasma outlet 154 prevent blood cells from passing through the membrane layer.

In one embodiment utilizing the asymmetric membrane as the blood separation membrane layer, the blood separation membrane layer is a single layer comprising a Vivid GR plasma separation membrane that is cut into a circular disk shape having a diameter of 12 mm holding a blood intake volume of, e.g., 35-40 μL, or a diameter of 25 mm holding a blood intake volume of, e.g., 140-160 μL, or diameter of 40 mm holding a blood intake volume of, e.g., 550-650 μL. The capacity of the blood separation membrane layer is 40-50 μL/cm².

Other blood separation devices may use uniform pore filters and/or glass microfiber-based blood cell retention membranes. Devices with uniform pore filters do not typically use a plurality of layers. These devices, which employ a single layer of uniform filters, typically experience membrane fouling and hemolysis due to a high concentration of blood cells in human whole blood. In devices that use glass fiber-based blood cell membranes as the blood separation membrane layer, the glass fiber-based membrane may trap blood cells on the surface and within a fiber mesh. Glass fiber-based membrane typically have large retention volumes and require external force for plasma recovery. While glass filter-based membrane layers may efficiently isolate blood cells from blood plasma without hemolysis, they also may not efficiently trap all blood cells and blood cells under applied pressure.

TABLE 1
Membrane Types

| Membrane | | Product | |
|---|---|---|---|
| type | Vendor | Name | Description |
| Uniform | Sterlitech | PCTE | Membrane has good wetability compared with |
| pore | | membrane | PES uniform pore membrane. Membrane |
| filter | | 1 um | fouling and hemolysis are observed when |
| paper | | | pressure applies. |
| | | PES | Membrane has higher void volume compared |
| | | membrane | with PCTE membrane. Membrane fouling and |
| | | 1.2 um | hemolysis are observed due to the high blood |
| | | | cell counts in the single layer separation |
| | | | process. The membrane can be used as the |
| | | | bottom layer to prevent excess blood cell |
| | | | leaking. |
| Glass | Whatman | GF/DVA | Untreated glass fiber. With a membrane |
| fiber | | | thickness of 785 um, the membrane has high |
| membrane | | | void volume with capability to handle large |
| | | | volume of blood (93 mg/cm2). However, |
| | | | protein adsorption is a concern with this type of |
| | | | membrane. Plasma recovery ratio is low due to |
| | | | the high dead volume inside the membrane. |
| | | LF1 | Thin membrane design with thickness of |
| | | | 247 μm. This membrane is ideal for a multiple |
| | | | layer membrane system, in which this membrane will |
| | | | be put as the top or medium layer for blood cell |
| | | | trapping and plasma separation. It needs to |
| | | | work with other membrane layer for plasma |
| | | | recovery and prevent blood cell leaking. |
| | | MF1 | The membrane has similar property with LF1, |
| | | | with higher blood separation capability. It can |
| | | | be used for process requires higher blood |
| | | | volume input. |
| | | VF2 | Membrane has property similar to GF/VDA |
| | | | membrane. |
| | | Fusion 5 | Similar to LF1, the membrane may be |
| | | | advantageous for multiple layer membrane |
| | | | separation system. |
| Asymmetric | Pall | Vivid-GF | Polyethersulfone (PES) based asymmetric |
| membrane | | | membrane that allows blood cells to be |
| | | | captured in the larger pores without lysing, while |
| | | | the plasma flows down into smaller pores on the |
| | | | downstream side of the membrane. This |
| | | | membrane is uncoated with capability of |
| | | | treating 20-30 μL blood per cm². |
| | | Vivid-GX | This membrane is similar to Vivid-GF. With a |
| | | | chemical coating process, this membrane |
| | | | performs better in preventing hemolysis and |
| | | | protein binding. |
| | | Vivid-GR | Compared with Vivid-GF and GX, this |
| | | | membrane has higher void volume, allowing for |
| | | | 40-50 μL blood volume input per cm². This may |
| | | | be advantageous for single layer or multiple |
| | | | layer separation system. |
| | Cobetter | HD B1B | The membrane has similar structure and |
| | | | treatment capability like Vivid-GR and can be |
| | | | used for both single layer or multiple layer |
| | | | separation system. |
| | FortisLife | Primecare SG | The membrane has similar property like Vivid- |
| | | | GX and can be used with single/multiple layer |
| | | | separation system. |
| | | Primecare X | The membrane has a thin membrane thickness |
| | | | with reduced capability for blood plasma |
| | | | separation. However, it is ideal to utilizing this |
| | | | membrane as the bottom layer in the multiple |
| | | | membrane system for plasma collection and |
| | | | stop blood cell leaking. |

With continued reference to FIG. 5, the sample collection receptacle 104 further includes a hydrophilic guide member 106 coupled to the plasma outlet 154 for facilitating the extraction of plasma from the plasma separation chamber 152, a flange 156 forming part of the plasma outlet 154 for selectively receiving the collection vial 108, and a plurality of recesses 148 formed on the main body 150 adjacent the blood inlet 140. In some embodiments of the sample collection receptacle 104, the blood inlet 140 and the plasma outlet 154 reside on opposite sides of the plasma separation chamber 152.

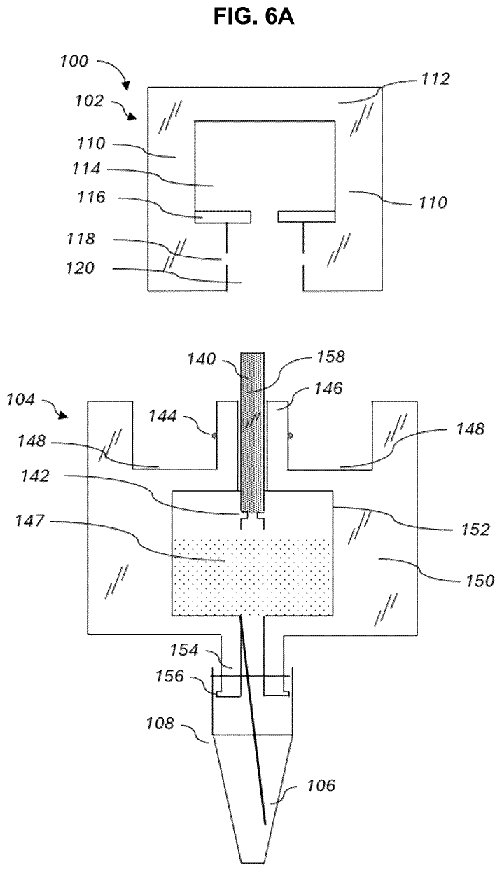

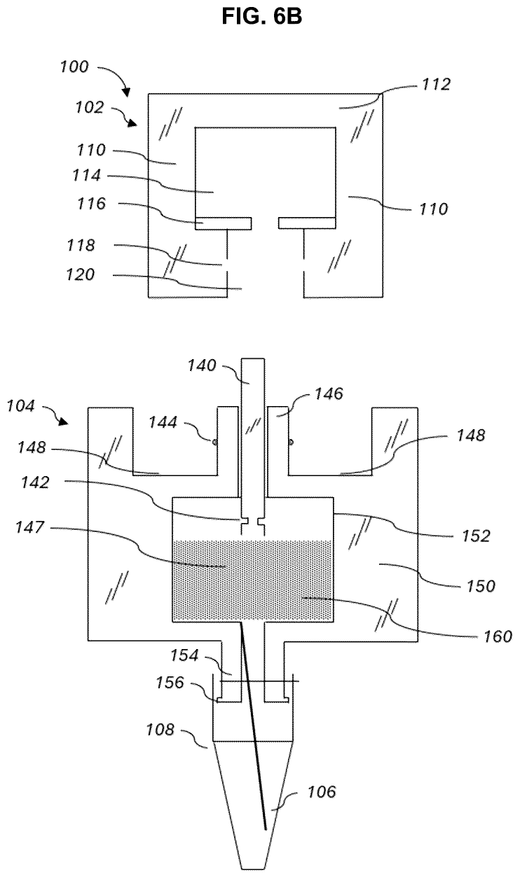

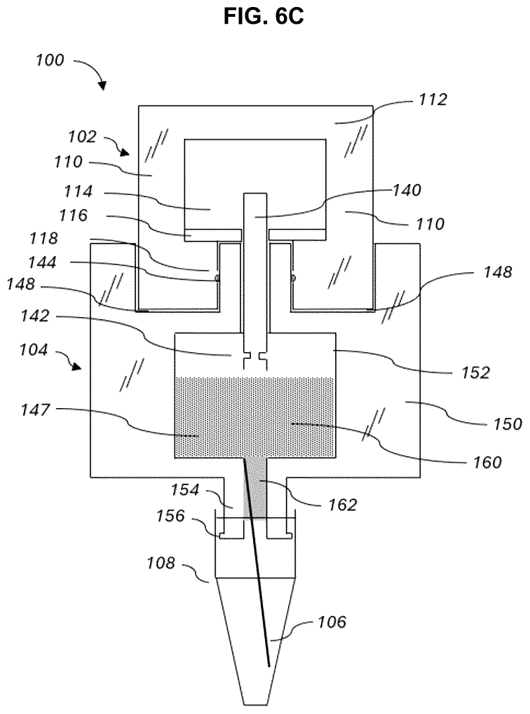

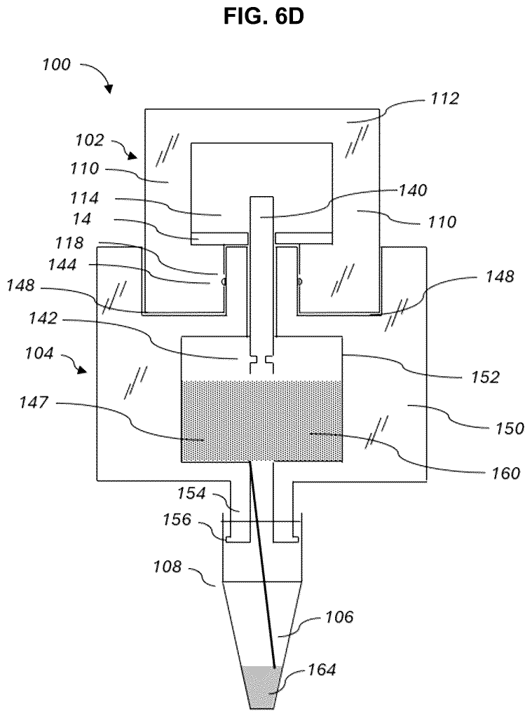

In other embodiments, the hydrophilic guide member 106 is a film strip, a tube or a combination thereof. In yet other embodiments, the hydrophilic guide member 106 may be adhered to an inner wall of the plasma outlet 154 directly underneath the plasma separation medium 146 and guide a plasma flow 162 (FIG. 6C) from the blood separation membrane layer to the collection vial 108. The hydrophilic surface of the hydrophilic guide member 106 may be easily wetted by an initial plasma volume flowing from the plasma outlet 154 and subsequently draw the remaining plasma to the bottom of the collection vial 108 with the aid of gravity and the free energy change ΔE provided by wetting the surface of the hydrophilic guide member 106, according to the equation:

ΔE=−γ·cos θ·ΔA,

where ΔA is the solid-vapor surface area on the strip. The hydrophilic surface provides a θ<90°, which creates a negative free energy change to draw the separated plasma spontaneously to the bottom of the collection vial 108 in a volume of separated plasma or plasma sample 164 (FIG. 6D).

With yet continued reference to FIG. 5, the sealing cap 102 defines a main body 112 including a pressure chamber 114 partially enclosed by the main body 112, and a plurality of legs 110 extending from the main body 112 that form an opening 120 therebetween that is in fluid communication with the pressure chamber 114. The sealing cap 102 further includes an annular spacer 116 disposed within the pressure chamber 114 adjacent the opening 120, and an O-ring groove 118 for selectively mating with an O-ring 144 disposed on the post 146 to form a seal between the cap 102 and the main body 150 of the sample collection receptacle 104. The cap 102 is configured to be selectively coupled to the sample collection receptacle 104 by inserting each of the plurality of legs 110 into one of the plurality of recesses 148 on the main body 150 of the sample collection receptacle 104. When the sealing cap 102 is coupled to the sample collection receptacle 104, the cap 102 is configured to apply an active, positive, and constant pressure to drive plasma flow and accelerate its separation from the whole blood sample inserted into the blood inlet 140, while leaving blood cells trapped within the plasma separation medium 147.

To exert positive pressure onto the sample collection receptacle 104, an internal volume of the pressure chamber 114 is compressed by coupling the cap 102 to the main body 150 of the sample collection receptacle 104. When the cap 102 is not coupled to the sample collection receptacle 104, the pressure chamber 114 fills with ambient air at the standard atmospheric pressure (e.g., 14.7 psi). As the cap 102 is coupled to the main body 150, a seal is formed between the main body 150 and the cap 102 via the O-ring 144 on the post 146 and the O-ring groove 118 on the cap 102. This seal traps the ambient air within the pressure chamber 114 and compresses the air to 83% of its original volume, thereby temporarily increasing the overall pressure in the pressure chamber 114 to 17.6 psi. The pressure chamber 114 then transfers this compressed air through the blood inlet 140 and into the plasma separation chamber 152. This change in pressure (ΔP) creates a pressure change of about 3 psi between the plasma separation chamber 152, which is temporarily experiencing an internal pressure of 17.6 psi, and the plasma outlet 154 that is at atmospheric pressure, or 14.7 psi. This change in pressure helps aid in the separation of plasma from the whole blood within the plasma separation medium 147, and actively forces separated plasma to flow out of the plasma separation medium 146 to accelerate the separation process significantly compared to a passive separation method. To control the amount of pressure exerted by the cap 102 and through the blood inlet 140 into the plasma separation chamber 152, the annular spacer 116 provided within the pressure chamber 114 acts as a stop when the cap 102 is coupled to the sample collection receptacle 104, by abutting a top surface of the post 146 located on the sample collection receptacle 104. By abutting the post 146, the annular spacer 116 ensures that a fixed volume of air within the pressure chamber 114 will be compressed each time the cap 102 is coupled to the sample collection receptacle 104, thereby improving accuracy and repeatability of plasma separation.

In some embodiments, to obtain cell-free and contamination-free plasma, a pressure at 1.07-1.2× of the atmospheric pressure is used, offering a relatively large tolerance on operating the device 100 and a sufficient force to rapidly complete a blood and plasma separation in less than 15 seconds. This leads to a volume reduction of the air within the pressure chamber 114 by compressing the air to 0.83-0.93× of the original volume of the pressure chamber 114, according to Boyle's Law where PV=constant and P is the gas pressure and Vis gas volume.

FIG. 6A-6B illustrate a step-by-step method for a user to take when using the device 100 to separate plasma from whole blood. First, the user inserts the collection vial 108 onto the flange 156 of the sample collection receptacle 104 to allow the separated plasma to be collected in the vial 108. In some embodiments, the device 100 can be used without the collection vial 108. Next, the user introduces a blood sample into the blood inlet 140 to fill the blood inlet 140 to a predefined fixed volume 158 (FIG. 6A) within the blood inlet 140. The user can use any method known in the art to fill the blood inlet 140, such as by capillary action, using a dropper, a pipette, etc. Then, to allow the blood sample to flow from the blood inlet 140 into the plasma separation chamber 152, the fluid regulator 142 needs to be actuated (e.g., opened) to permit fluid flow. In the illustrated embodiment, the user can actuate the fluid regulator 142 by applying a force (e.g., a tap from the user on a hard surface, or with a finger) to either the collection vial 108, or any other part of the sample collection receptacle 104. In other embodiments, the fluid regulator 142 allows the blood sample to slowly flow through the regulator 142 and into the plasma separation chamber 152 in response to gravity and capillary action, and the user does not need to manually open the fluid regulator 142 by applying a force. Once, the user applies the force to open the fluid regulator 142, the fixed volume 158 of blood flows through the fluid regulator 142 and into the plasma separation chamber 152 to fully absorb into the separation medium 146 until the medium is a saturated medium 160. When the separation medium 146 becomes saturated, the user then couples the cap 102 to the sample collection receptacle 104 to facilitate the separation of plasma from the whole blood sample by biasing the blood sample through the separation medium 146 and into the collection vial 108 utilizing the positive pressure applied by the cap 102.

To couple the cap 102 to the sample collection receptacle 104, the user aligns the opening 120 in the cap 102 with the blood inlet 140 and inserts each of the legs 110 of the cap 102 into one of the plurality of recesses 148 formed on the main body 150 of the sample collection receptacle 104. When the user couples the cap 102 to the sample collection receptacle 104 (FIG. 6C), a seal is formed between the cap 102 and the sample collection receptacle 104 via the O-ring 144 disposed on the post 146 and the O-ring groove 118 on the cap 102, thereby pressurizing the air within the pressure chamber 114. This pressure creates a change in pressure between the pressure chamber 114 and the plasma outlet 154 that biases the blood sample through the plasma separation membrane 146 and down the hydrophilic guide member 106 into the collection vial 108 as separated plasma 162 (FIG. 6C). The separated plasma 162 then collects at the bottom of the collection vial 108 as a plasma sample 164 (FIG. 6D) ready for analysis by the user. After the plasma sample 164 is collected, the user can then remove the collection vial 108 from the flange 156 and close the vial 108 with the cover 109 (FIG. 1) to preserve the plasma sample 164 for later analysis.

In some embodiments, the sample collection receptacle 104 and the collection vial 108 are disposable and are intended for a single use, whereas the cap 102 can be reused if properly operated without contamination.

In other embodiments, the plasma outlet 154 is designed to maximize plasma collection from the blood sample, by utilizing a zero-dead-volume interface between the collection vial 108 and the plasma outlet 154 for increasing the capillary pressure, thereby resulting in a high resistance for plasma flow. In yet other embodiments, the plasma outlet 154 includes an interface partially forming the plasma outlet 154 for facilitating fluid communication with an analytical device that can replace the collection vial 108 to identify certain characteristics of the plasma sample 164.

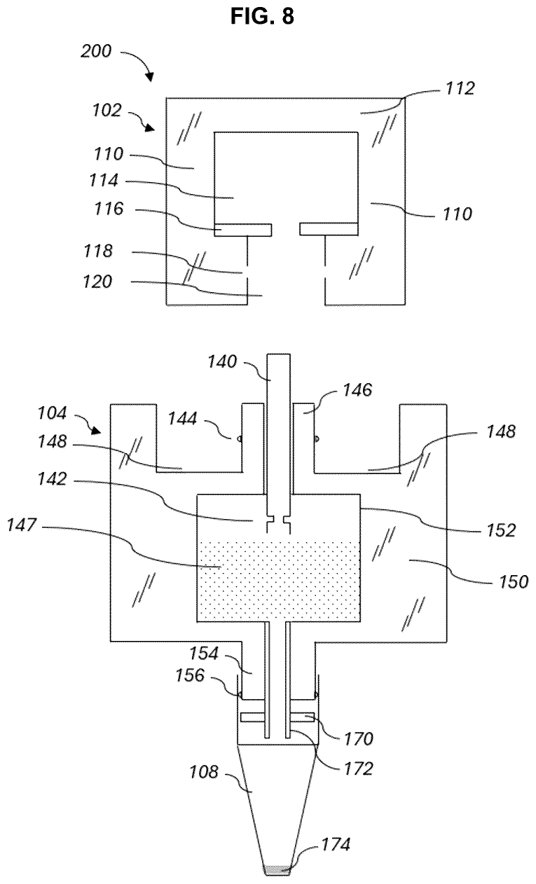

FIG. 8 illustrates an alternative embodiment of a fluid separation device 200 including like components and features of the fluid separation device 100, with differences explained below. In some embodiments, the fluid separation device 200 is configured to separate plasma from whole blood. The fluid separation device 200 includes the sample collection receptacle 104, the sealing cap 102, and a collection vial 108. The collection vial 108 includes an annular holder 170 integrally formed with the collection vial 108 that holds a hydrophilic guide member 172. In the illustrated embodiment, the hydrophilic guide member 172 is a tube including a hydrophilic surface configured to allow plasma collection and storage under capillary pressure. In some embodiments, the tube 172 includes an outer diameter of 1.65 mm. In other embodiments, the outer diameter of the tube 172 is between 0.3 mm and 3 mm.

In operation, the capillary pressure inside the collection tube 172 assists plasma movement from the plasma separation chamber 152 to the collection vial 108 by breaking through the capillary barrier in the plasma separation medium 147. After the plasma separation process, and the user detaches the collection vial 108 from the sample collection receptacle 104, the tube 172 is disconnected from the plasma separation medium 147, and the plasma is retained inside the tube 172. To prevent backflow of the plasma into the plasma separation medium 126 during the detachment process, the internal surface of the tube 172 forms an acute angle. In other embodiments, the tube 172 includes holes positioned adjacent a top portion of the tube 172 to prevent backflow.

To collect separated plasma in the collection vial 108, the user follows the same steps as detailed above with regard to the first embodiment of the fluid separation device 100, with the additional step of applying a force (e.g., tapping the vial 108 on a hard surface) to the collection vial 108 once the plasma has collected in the tube 172. Once this force is applied, the plasma sample will flow from the tube 172 and collect at the bottom of the vial 108.

In some embodiments, the collection vial 108 includes a solid bottom or a foil sealed bottom 174 which can be punched through to release the collected plasma sample to a downstream assay device.

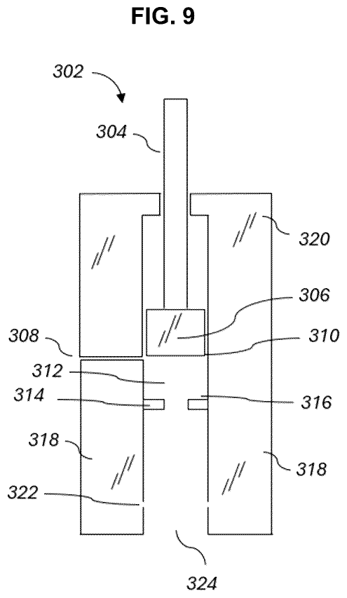

FIG. 9 illustrates an alternative embodiment of a sealing cap 302 for use with either of the fluid separation devices 100, 200. The sealing cap 302 defines a main body 320 including a plurality of legs 318 extending from the main body 320 forming an opening 324 therebetween, a plunger 304 having a plunger head 306 that is slidably coupled to the main body 320, and a pressure chamber 312 partially enclosed by the main body 320. When the cap 302 is not coupled to the sample collection receptacle 104, the pressure chamber 312 is open to atmospheric pressure via a vent 308 formed in the main body 320 of the cap 302 and the opening 324. The cap 302 further includes an O-ring groove 322 located adjacent the opening 324 for selectively receiving the O-ring 144 on the post 146 (FIG. 5) of the sample collection receptacle 104 to form a seal therebetween, and an annular stop 314 mounted within the main body 320 adjacent the pressure chamber 312 for selectively inhibiting a travel path of the plunger 304 within the main body 320.

To couple the cap 302 to the sample collection receptacle 104, the user aligns the opening 324 with the post 146 or blood inlet 140 (FIG. 5) on the sample collection receptacle 104, and then inserts each of the legs 318 into respective recesses 148 of the sample collection receptacle 104. Unlike the sealing cap 102, once the cap 302 is coupled to the sample collection receptacle 104, the pressure chamber 312 is not automatically compressed by the seal formed between the cap 302 and the sample collection receptacle 104 due to the vent 308. Rather, the user must manually depress the plunger 304 along its travel path to generate the positive change in pressure between the pressure chamber 312 and the sample collection receptacle 104. To generate the positive pressure, the user depresses the plunger 304 from a starting position 310 until the plunger head 306 contacts the annular stop 314 at an ending position 316. This movement compresses the air within the compression chamber 312 to generate the positive pressure needed to initiate the plasma separation within the sample collection receptacle 104. The annular stop 314 operates as a quality control measure to ensure the travel path of the plunger 304 is the same every time the plunger 304 is actuated, thereby ensuring that the positive pressure applied to sample collection receptacle 104 is consistently accurate and repeatable. In some embodiments, the plunger 304 automatically travels along its travel path by actuating a switch.

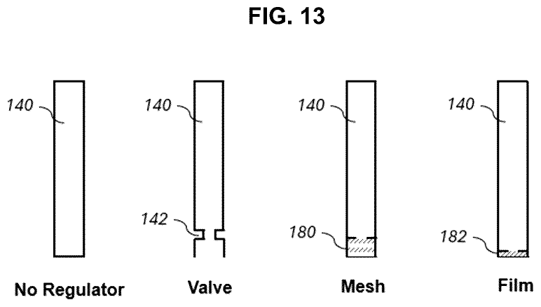

FIG. 13 illustrates alternative embodiments of fluid regulators for use with any of the devices 100, 200. In some embodiments, the device 100, 200 does not include a fluid regulator. In other embodiments, the fluid regulator is a capillary valve 142 defining an opening measuring between 0.5-2 mm. The capillary valve 142 is configured to temporarily hold the fluid sample (e.g., blood sample) before the sample can enter the receptacle 104. In other embodiments, the fluid regulator is a mesh 180 including a plurality of hydrophobic pores ranging from 50-200 micrometers (μm) that are configured to retain the fluid sample before the sample can enter the receptacle 104. In other embodiments, the fluid regulator is a film 182 configured to rupture under an applied force. For example, after applying a load or force to the device 100, 200 (e.g., a tap from the user on a hard surface, or with a finger), the film 182 is configured to rupture to allow the fluid sample to enter the sample collection receptacle 104. In some embodiments, the film 182 can be ruptured by a medical implement, such as a needle tip. While each of the illustrated fluid regulators 142, 180, 182 are shown as being disposed on a lowermost portion of the blood inlet 140, each of the fluid regulators 142, 180, 182 may be positioned elsewhere on the blood inlet 140.

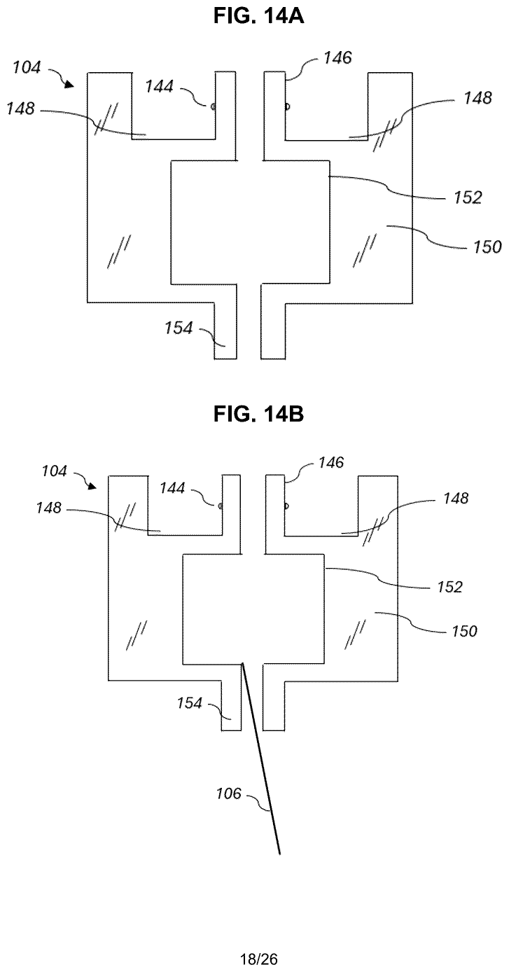

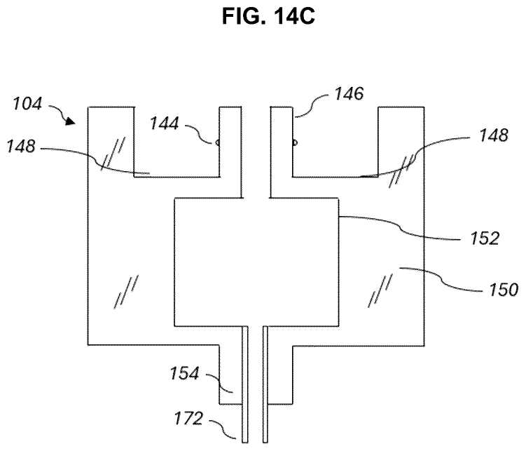

FIGS. 14A-14C illustrate alternative embodiments of hydrophilic guide members for use with any of the devices 100, 200. In one embodiment, the device 100, 200 does not include a hydrophilic guide member coupled to the plasma outlet 154. In other embodiments, the hydrophilic guide member is a film 106 configured to facilitate fluid flow, e.g., plasma flow 162, from the receptacle 104 and into the collection vial 108. In some embodiments, the film 106 is also configured to leave minimal fluid residue (e.g., plasma residue) on a surface of the film 106 as compared to other methods. In other embodiments, the hydrophilic guide member is a tube 172 configured to facilitate fluid flow, e.g., plasma flow 162, from the receptacle 104 and into the collection vial 108.

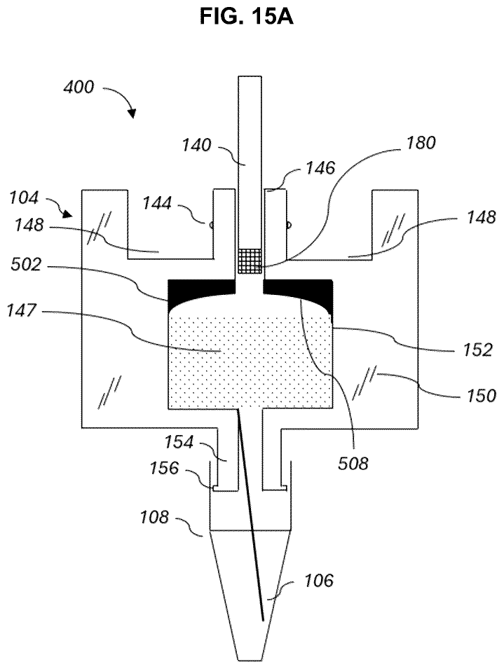

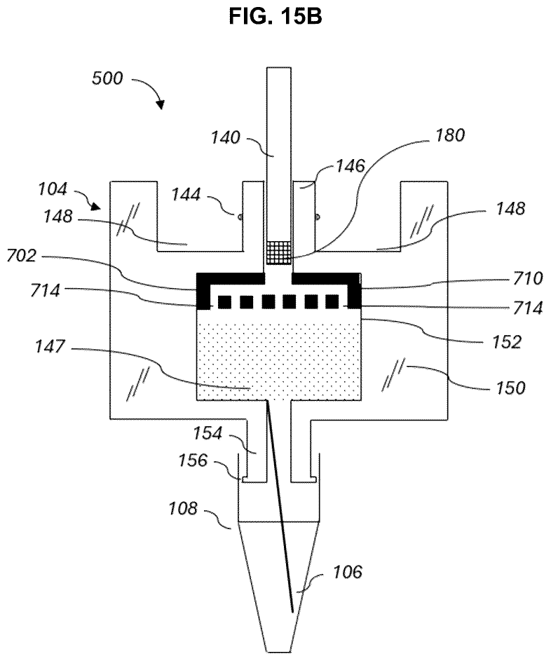

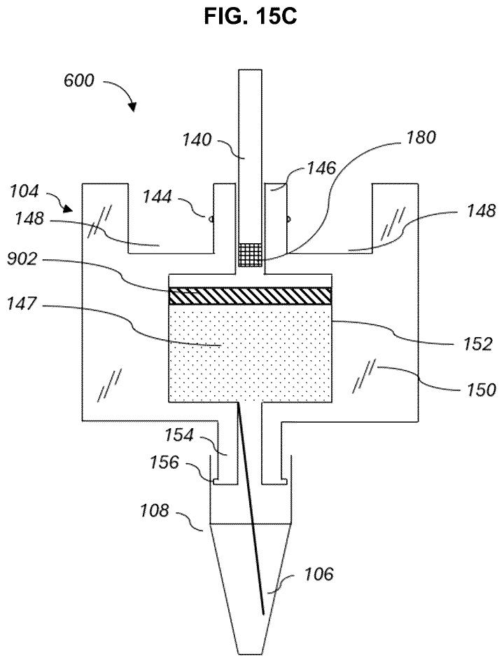

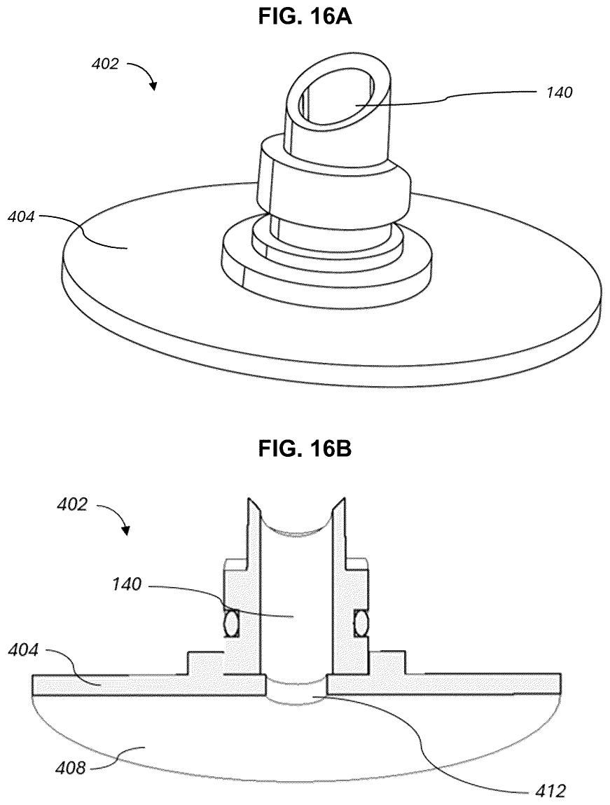

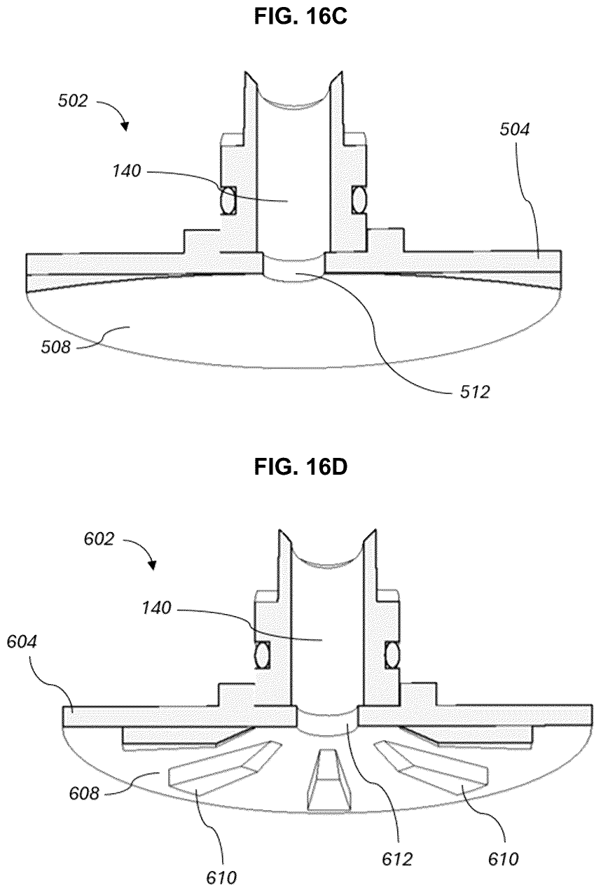

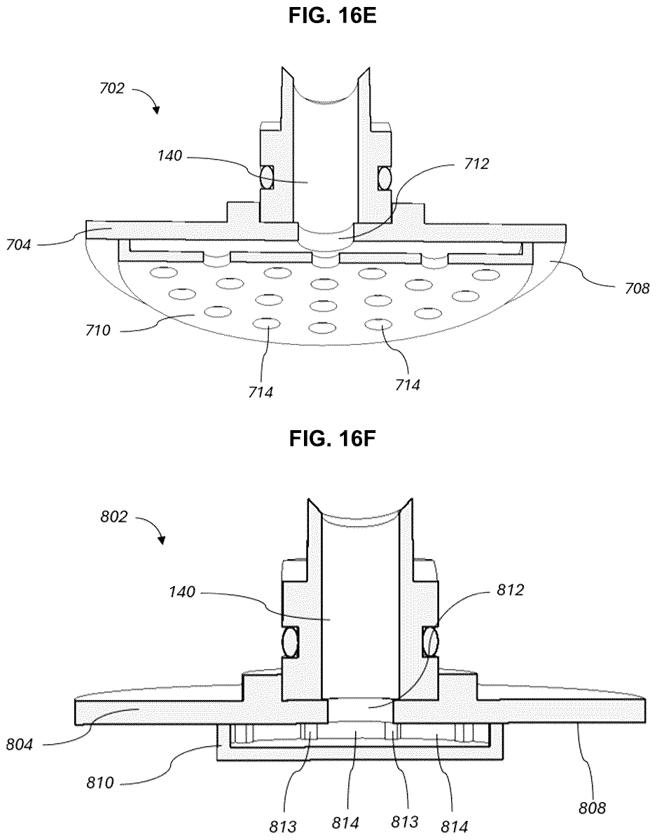

FIGS. 15A-15C illustrate alternative embodiments of the device 100, 200. FIG. 15A includes like components and features of the device 100, with differences explained below. FIG. 15A illustrates an alternative embodiment of a fluid separation device 400. The fluid separation device 400 includes a blood inlet 140 having a fluid regulator 180 configured to control or regulate aspects of fluid within the inlet 104, such as fluid flow, etc. The fluid regulator 180 illustrated is a mesh including a plurality of hydrophobic pores ranging from 50-200 micrometers (μm) that are configured to retain the fluid sample before the sample can enter the receptacle 104. The fluid separation device 400 also includes a fluid distributor 502. The fluid distributor 502 is in fluid communication with the blood inlet 140 and the plasma separation chamber 152. The fluid distributor 502 is configured to guide a flow direction of the fluid sample and promote substantially uniform distribution of the fluid sample across a surface of the plasma separation medium 147. The illustrated fluid distributor 502 is positioned within the plasma separation container 152 adjacent the fluid regulator 180 and upstream from the plasma separation medium 147. The fluid distributor 502 may define an internal surface of the plasma separation chamber 152. The illustrated fluid distributor 502 includes a body 504 (FIG. 16C) defining an outer surface 508. The illustrated outer surface 508 is curved and defines an inner surface of the plasma separation chamber 152. The device 400 also includes an aperture 512, which facilitates fluid communication between the blood inlet 140 and the plasma separation chamber 152. During use, fluid from the sample is configured to enter the chamber 152 through the aperture 512 and be dispersed throughout the chamber 152 by the curved outer surface 408.

FIG. 15B illustrates an alternative embodiment of a fluid separation device 500 utilizing another embodiment of a fluid distributor 702. FIG. 15B includes like components and features of the device 400, with differences explained below. The fluid distributor 702 is in fluid communication with the blood inlet 140 and the plasma separation chamber 152. The illustrated fluid distributor 702 is positioned within the plasma separation container 152 adjacent the fluid regulator 180 and upstream from the plasma separation medium 147. The illustrated fluid distributor 702 includes a body 704 (FIG. 16E) defining an outer surface 708. The illustrated fluid distributor 702 also includes a manifold 710, and an aperture 712 for facilitating fluid communication between the blood inlet 140 and the manifold 710. The manifold 710 includes a plurality of ports 714 that are configured to divide and evenly distribute the fluid sample through the plasma separation chamber 152 for absorption in the medium 147. In some embodiments, each of the ports 714 permit fluid flow in a vertical direction.

FIG. 15C illustrates an alternative embodiment of a fluid separation device 600 utilizing another embodiment of a fluid distributor 902. FIG. 15C includes like components and features of the device 400, with differences explained below. The fluid distributor 902 is in fluid communication with the blood inlet 140 and the plasma separation chamber 152. The illustrated fluid distributor 702 is positioned within the plasma separation chamber 152 and between the plasma separation medium 147 and the fluid regulator 180. The illustrated fluid distributor 902 includes an insert configured to receive the fluid sample prior to contacting the medium 147. In other embodiments, the fluid sample spreads throughout the distributor 902 or along an interface between the distributor 902 and the medium 147. In some embodiments, the insert may be composed of a hydrophilic film, porous membrane, mesh, or combinations thereof, and may be configured to promote fluid flow via capillary action.

In some embodiments, each of the fluid distributors 402, 702, 902 can be positioned within the plasma separation chamber 152 such that they are spaced from the medium 147 and are directing the fluid sample from the inlet 140 across the medium 147. In other embodiments, each of the fluid distributors 402, 70, 902 can be positioned within the separation chamber 152 such that they are contacting the medium 147.

FIGS. 16A-16F illustrate alternative embodiments of a fluid distributor for use with any of the devices 100, 200, 400, 500, 600. Each of the fluid distributors described below are configured to function similar to the fluid distributors 502, 702 described above. Since the fluid distributors 502, 702 have already been described in the abovementioned embodiments of the device 400, 500, those embodiments will not be redescribed here for sake of brevity.

FIGS. 16A and 16B illustrate a fluid distributor 402 including a body 404 defining an outer surface 408, and an aperture 412 configured to facilitate fluid communication between the blood inlet 140 and the distributor 402. In the illustrated embodiment, the body 404 is disc shaped and the surface 408 is a flat surface that defines an inner surface of the separation chamber 152.

FIG. 16D illustrates a fluid distributor 602 including a body 604 defining an outer surface 608 that has a plurality of ridges 610. In the illustrated embodiment, the ridges 610 are radially disposed about an aperture 612. Each of the ridges 610 is configured to evenly distribute the fluid sample throughout the chamber 152. The aperture 612, is configured to facilitate fluid communication between the blood inlet 140 and the distributor 602.

FIG. 16F illustrates a fluid distributor 802 including a body 804 defining an outer surface 808, and a manifold 810 coupled to the surface 808. The manifold 710 includes a plurality of posts 813 radially disposed about an aperture 812 formed on the body 804. Each of the posts 813 defines channels 814 or ports therebetween. Each of the channels 814 is configured to permit fluid flow, for example, for the fluid sample to flow through. In some examples, each of the channels 814 is configured to permit fluid flow in a horizontal direction. In the illustrated embodiment, the aperture 812 is configured to facilitate fluid communication between the blood inlet 140 and the distributor 802.

In some embodiments, each of the respective bodies of the fluid distributors 402, 502, 602, 702, and 802 are disc shaped like the body 404 of the fluid distributor 402. However, in other embodiments each of the respective bodies of the fluid distributors may be other shapes known in the art.

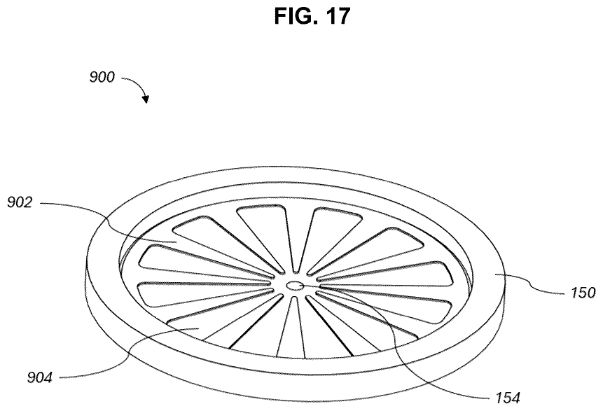

FIG. 17 illustrates a medium support 900 that can be implemented in any of the devices 100, 200, 400, 500, 600. The medium support 900 is configured to support the plasma separation medium 147. In some embodiments, the medium support 900 is disposed at a lower portion, or bottom of the plasma separation chamber 152 adjacent to the plasma outlet 154 and is configured to elevate the plasma separation medium 147 from a lower surface of the separation chamber 152. In other embodiments, the medium support 900 is integrated into the body 150 of sample collection receptacle 104. In some embodiments, the medium support 900 includes a plurality of ridges 902 and a plurality of channels 904 that are configured to guide the plasma toward the outlet 154. In the illustrated embodiment, the ridges 902 are radially disposed about the plasma outlet 154, and the channels 904 are defined between adjacent ridges 902. In some embodiments, each of the ridges 902 is configured to elevate the separation medium 147 above the channels 904 in order to facilitate fluid flow from the medium 147 through one or more of the channels 902 and into the outlet 154. In other embodiments, the ridges 902 and the channels 904 may be other shapes or configurations.

One embodiment described herein is a fluid separation device comprising: a sample collection receptacle for receiving a fluid sample, the sample collection receptacle including: an inlet for receiving the fluid sample, a separation chamber in fluid communication with the inlet, the separation chamber having a membrane to filter the fluid sample, an outlet in fluid communication with the separation chamber, and at least one hydrophilic guide member coupled to the outlet; and a cap selectively coupled to the sample collection receptacle, the cap including a pressure chamber in fluid communication with the inlet; wherein, when the cap is coupled to the sample collection receptacle, the pressure chamber exerts a compression force through the inlet to move the fluid sample through the membrane and into the outlet.

Another embodiment described herein is a fluid separation device comprising: a sample collection receptacle for receiving a fluid sample, the sample collection receptacle including: an inlet for receiving the fluid sample, a separation chamber in fluid communication with the inlet, the separation chamber having a membrane to filter the fluid sample, a fluid distributor configured to distribute the fluid sample across the membrane, wherein the fluid distributor comprises, an inner surface of the separation chamber, a manifold, or an insert, an outlet in fluid communication with the separation chamber, and at least one hydrophilic guide member coupled to the outlet; and a cap selectively coupled to the sample collection receptacle, the cap including a pressure chamber in fluid communication with the inlet; wherein, when the cap is coupled to the sample collection receptacle, the pressure chamber exerts a compression force through the inlet to move the fluid sample through the membrane and into the outlet. In one aspect, the fluid separation device further comprises: a flange partially forming the outlet of the sample collection receptacle; and a collection vessel removably coupled to the flange. In another aspect, the fluid separation device further comprises: an interface partially forming the outlet of the sample collection receptacle and facilitating fluid communication with an analytical device. In another aspect, the fluid separation device further comprises a fluid regulator for selectively permitting fluid communication between the inlet and the separation chamber. In another aspect, the fluid regulator is a capillary valve, a layer of mesh with pore size of 50 to 200 μm, or a thin film configured to rupture under an applied load. In another aspect, the fluid separation device further comprises a support disposed at a lower portion of the separation chamber, the support being configured to support the membrane, wherein the support facilitates fluid communication between the membrane and the outlet of the sample collection receptacle. In another aspect, the inlet is configured to contain a fixed volume of the fluid sample. In another aspect, when a force is applied to the device, the fluid distributor permits fluid communication between the inlet and the separation chamber by releasing the fixed volume of the fluid sample. In another aspect, the at least one hydrophilic guide member extends from the outlet of the sample collection receptacle substantially vertically in a direction of the compression force. In another aspect, the at least one hydrophilic guide member extends from the outlet of the sample collection receptacle at an angle of at least 10° from a horizontal plane perpendicular to a direction of the compression force. In another aspect, the at least one hydrophilic guide member comprises a hydrophilic material, the hydrophilic material comprising a liquid drop contact angle θ<65°. In another aspect, the inlet has a liquid drop contact angle θ<90°. In another aspect, the membrane has an average porosity of no less than 40%. In another aspect, the membrane at least has a minimum pore size of 0.1 to 2 μm.

Another embodiment described herein is a fluid separation device, comprising: a sample collection receptacle for receiving a fluid sample, the sample collection receptacle including: an inlet for receiving the fluid sample; a separation chamber in fluid communication with the inlet, the separation chamber having a membrane to filter the fluid sample; a fluid regulator for selectively permitting fluid communication between the inlet and the separation chamber; a fluid distributor to distribute the fluid sample across the membrane; an outlet in fluid communication with the separation chamber; and at least one hydrophilic guide member coupled to the outlet; and a cap selectively coupled to the sample collection receptacle, the cap including a pressure chamber in fluid communication with the inlet; wherein, when the cap is coupled to the sample collection receptacle, the pressure chamber exerts a compression force through the inlet to move the fluid sample through the membrane and into the outlet.

Another embodiment described herein is a method for separating a fluid sample comprising: applying a fluid sample to the fluid separation device described herein; exerting a force onto the device to permit a volume of the fluid sample to pass through a fluid regulator into the separation chamber; coupling the cap to the sample collection receptacle to exert a compression force; separating the fluid sample in the membrane of the separation chamber; and collecting a separated aliquot from the fluid sample via the outlet in fluid communication with the separation chamber. In one aspect, the fluid sample is a blood sample. In another aspect, the separated aliquot is plasma. In another aspect, the method further comprises: affixing a collection vessel to a flange of the outlet on the fluid separation device. In another aspect, the separated aliquot is collected in about 30-180 seconds.

Another embodiment described herein is a kit comprising: one or more the fluid separation devices described herein; a means for obtaining a blood sample from a human or animal subject; optionally, a blood collection vial; optionally, one or more antiseptics; optionally one or more pairs of gloves; and packaging, labels, and instructions for use.

Another embodiment described herein is the use of the fluid separation device described herein to separate plasma from a blood sample.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. A methods may be performed in any logical order and various aspects may be performed simultaneously or sequentially. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A fluid separation device comprising:
   a sample collection receptacle for receiving a fluid sample, the sample collection receptacle including:
      an inlet for receiving the fluid sample,
      a separation chamber in fluid communication with the inlet, the separation chamber having a membrane to filter the fluid sample,
      a fluid distributor configured to distribute the fluid sample across the membrane, wherein the fluid distributor comprises, an inner surface of the separation chamber, a manifold, or an insert,
      an outlet in fluid communication with the separation chamber, and
      at least one hydrophilic guide member coupled to the outlet; and
   a cap selectively coupled to the sample collection receptacle, the cap including a pressure chamber in fluid communication with the inlet;
      wherein, when the cap is coupled to the sample collection receptacle, the pressure chamber exerts a compression force through the inlet to move the fluid sample through the membrane and into the outlet.
Clause 2. The fluid separation device of clause 1, further comprising:
   a flange partially forming the outlet of the sample collection receptacle; and
   a collection vessel removably coupled to the flange.
Clause 3. The fluid separation device of clause 1 or 2, further comprising:
   an interface partially forming the outlet of the sample collection receptacle and facilitating fluid communication with an analytical device.
Clause 4. The fluid separation device of any one of clauses 1-3, further comprising a fluid regulator for selectively permitting fluid communication between the inlet and the separation chamber.
Clause 5. The fluid separation device of clause 4, wherein the fluid regulator is a capillary valve, a layer of mesh with pore size of 50 to 200 μm, or a thin film configured to rupture under an applied load.
Clause 6. The fluid separation device of any one of clauses 1-5, further comprising a support disposed at a lower portion of the separation chamber, the support being configured to support the membrane, wherein the support facilitates fluid communication between the membrane and the outlet of the sample collection receptacle.
Clause 7. The fluid separation device of any one of clauses 1-6, wherein the inlet is configured to contain a fixed volume of the fluid sample.
Clause 8. The fluid separation device of clause 7, wherein, when a force is applied to the device, the fluid distributor permits fluid communication between the inlet and the separation chamber by releasing the fixed volume of the fluid sample.
Clause 9. The fluid separation device of any one of clauses 1-8, wherein the at least one hydrophilic guide member extends from the outlet of the sample collection receptacle substantially vertically in a direction of the compression force.
Clause 10. The fluid separation device of any one of clauses 1-9, wherein the at least one hydrophilic guide member extends from the outlet of the sample collection receptacle at an angle of at least 10° from a horizontal plane perpendicular to a direction of the compression force.
Clause 11. The fluid separation device of any one of clauses 1-10, wherein the at least one hydrophilic guide member comprises a hydrophilic material, the hydrophilic material comprising a liquid drop contact angle θ<65°.
Clause 12. The fluid separation device of any one of clauses 1-11, wherein the inlet has a liquid drop contact angle θ<90°.
Clause 13. The fluid separation device of any one of clauses 1-12, wherein the membrane has an average porosity of no less than 40%.
Clause 14. The fluid separation device of any one of clauses 1-13, wherein the membrane at least has a minimum pore size of 0.1 to 2 μm.
Clause 15. A method for separating a fluid sample comprising:
   applying a fluid sample to the fluid separation device of any one of clauses 1-14;
   exerting a force onto the device to permit a volume of the fluid sample to pass through the fluid regulator into the separation chamber;
   coupling the cap to the sample collection receptacle to exert a compression force;
   separating the fluid sample in the membrane of the separation chamber; and
   collecting a separated aliquot from the fluid sample via the outlet in fluid communication with the separation chamber.
Clause 16. The method of clause 15, wherein the fluid sample is a blood sample.
Clause 17. The method of clause 15, wherein the separated aliquot is plasma.
Clause 18. The method of any one of clauses 15-17, further comprising:
   affixing a collection vessel to a flange of the outlet on the fluid separation device.
Clause 19. The method of any one of clauses 15-18, wherein the separated aliquot is collected in about 30-180 seconds.
Clause 20. A kit comprising:
   one or more fluid separation devices of any one of clauses 1-14,
   a means for obtaining a blood sample from a human or animal subject;
   optionally, a blood collection vial;
   optionally, one or more antiseptics;
   optionally one or more pairs of gloves; and
   packaging, labels, and instructions for use.
Clause 21. Use of the fluid separation device of any one of clauses 1-14 to separate plasma from a blood sample.

### EXAMPLES

### Example 1

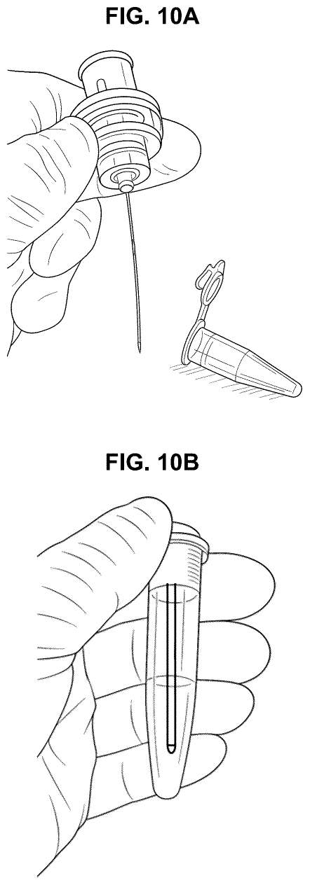

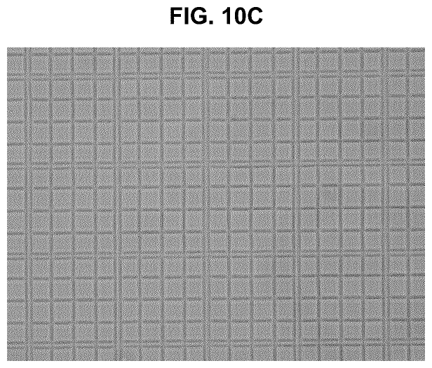

Examples of the plasma separated from the plasma separation apparatus show the quality of the plasma are clear and cell-free. FIG. 10A-C shows the plasma on the collection film guide (FIG. 10A), in a collection tube (FIG. 10B), and after dispensing the plasma on a hemacytometer (FIG. 10C). The plasma on the hemacytometer has no white and red blood cells observed, demonstrating the effectiveness of the plasma separation apparatus in separating the plasma from blood cells.

### Example 2

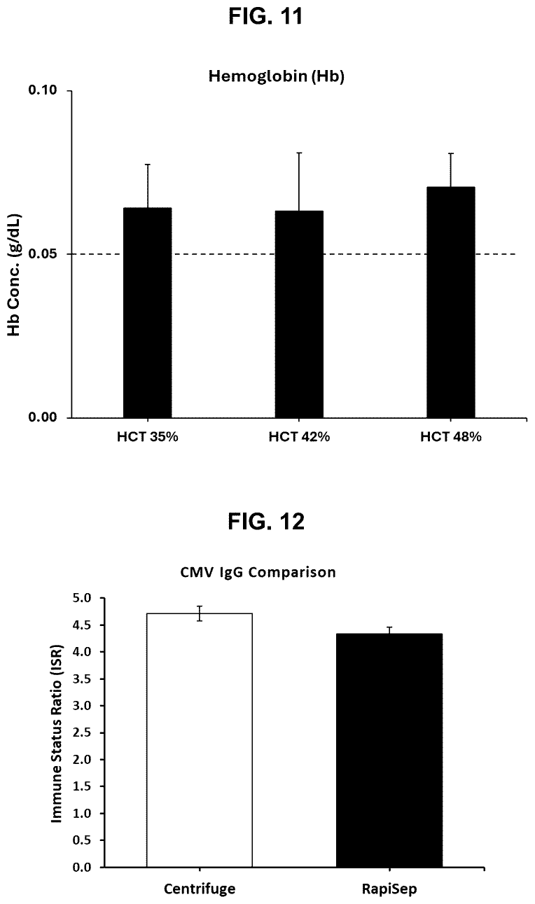

Table 2 shows a representative data sheet acquired by the plasma separation apparatus to demonstrate the quality of the separated plasma, including the blood consumption volume, plasma collection volume, white, red and platelet cell counts in the plasma, and the hemoglobin (Hb) value of the plasma. This data demonstrated a plasma yield of 30% with no large white and red blood cells or small platelet cells in the plasma that could potentially interfere with a downstream assay. The clearness of plasma is measured by hemoglobin value compared to the plasma separated using a centrifugation method (FIG. 11). The plasma separation apparatus shows no bias on blood samples with varied hematocrits in minimizing cell lysis during the separation process, producing plasma with Hb value close to 0.05 g/dL of centrifuged plasma (dashed line).

TABLE 2
RapiSep Performance

| Parameter | Value |
|---|---|
| Separation Time (sec) | 30 |
| RBC + WBC (number/μL) | 0 |
| Platelets (number/μL) | 0.1 |
| Hemoglobin (g/dL) | 0.1 |

### Example 3

FIG. 12 demonstrates experiments performed using plasma separated using the plasma separation apparatus described herein (“RapiSep”). FIG. 12 shows an antibody assay using plasma separated from this apparatus is comparable to plasma separated from a standard centrifugation method. The assay was performed according to the Captia™ Cytomegalovirus (CMV) IgG ELISA Kit instructions (Trinity Biotech). An ISR value >1.10 indicates a presence of detectable antibody, indicative of current or previous infection of CMV. Comparison between the two plasma samples shows there was no significant difference (p=0.08, two-tailed T-test) between plasma separated using the plasma separation device described herein and a standard method for separating plasma. This shows that plasma separated using the plasma separation device described herein can be used for clinical-relevant diagnostic testing.

### Example 4

Various materials were implemented into the outlet 154 (Table 3). The impact of the various materials on the separation time was measured. The results showed that the use of a hydrophobic surface had a separation time to 120 seconds. In contrast, a hydrophilic surface exhibited a separation time of 10 seconds. The results obtained with the hydrophobic surface may occur due to an increase the capillary pressure, which may result in a high resistance for plasma flow.

TABLE 3
Outlet Materials and Blood Sample Separation Times

| Material | Surface | Description |
|---|---|---|
| Grey Resin | hydrophobic | >1 minute for plasma to overcome a short |
| | | capillary barrier and is collected inside |
| | | the collection vial |
| Durable | hydrophobic | >3 minutes for plasma to overcome a short |
| Resin | | capillary barrier, and less volume is |
| | | collected due to the surface unevenness |
| PMMA | hydrophobic | >1 minute for plasma to overcome a short |
| | | capillary barrier and are collected inside |
| | | the collection vial |
| Tube 1 | hydrophobic | Plasma barely enters into the tube |
| Tube 2 | hydrophilic | Plasma collection complete within 10 sec |
| Film 1 | hydrophobic | Plasma can accumulate on the top of this |
| | | film forming a drop but is reluctant to |
| | | continue flowing down to the collection |
| | | vial |
| Film 2 | hydrophilic | Approximately 10 to 20 sec for plasma to |
| | | accumulate on the film and then collection |
| | | within 10 sec |

### Example 5

FIG. 13 shows examples of different fluid regulators. In one aspect, the fluid regulator is a valve, a mesh, a film, or the like. The fluid regulator is configured to temporarily hold a volume of input blood prior to actuation of the fluid regulator to release the blood into the separation chamber.

### Example 6

The plasma separation apparatus described herein (RapiSep) is scalable to handle various input blood volumes to produce different volumes of plasma for different applications. The plasma volume is proportional to blood input volume, and the yield is about 30%. Three representative sizes of the RapiSep are shown in Table 4, covering a plasma volume range from ~10 μL to sub-milliliter, which enables plasma volume requirements for most diagnostic assays.

TABLE 4
RapiSep Sizes

| Parameter | S (μL) | M (μL) | L (mL) |
|---|---|---|---|
| Blood Input | 35 | 150 | 0.6 |
| Plasma Output | 12 | 50 | 0.2 |

### Example 7

Examples of the hydrophilic guide member could be a thin film or a tube (FIGS. 14B and 14C), which facilitates the plasma being pulled out from the receptacle 104 to a collection vial 108. The thin film leaves no plasma residue as compared to the tube option, which may leave plasma residue on the inside of the tube and trap a portion of plasma volume inside the tube.

What is claimed:

1. A fluid separation device comprising:
a sample collection receptacle for receiving a fluid sample, the sample collection receptacle including:
an inlet for receiving the fluid sample,
a separation chamber in fluid communication with the inlet, the separation chamber having a membrane to filter the fluid sample,
a fluid distributor configured to distribute the fluid sample across the membrane, wherein the fluid distributor comprises, an inner surface of the separation chamber, a manifold, or an insert,
an outlet in fluid communication with the separation chamber, and
at least one hydrophilic guide member coupled to the outlet; and
a cap selectively coupled to the sample collection receptacle, the cap including a pressure chamber in fluid communication with the inlet;
wherein, when the cap is coupled to the sample collection receptacle, the pressure chamber exerts a compression force through the inlet to move the fluid sample through the membrane and into the outlet.

2. The fluid separation device of claim 1, further comprising:
a flange partially forming the outlet of the sample collection receptacle; and
a collection vessel removably coupled to the flange.

3. The fluid separation device of claim 1, further comprising:
an interface partially forming the outlet of the sample collection receptacle and facilitating fluid communication with an analytical device.

4. The fluid separation device of claim 1, further comprising a fluid regulator for selectively permitting fluid communication between the inlet and the separation chamber.

5. The fluid separation device of claim 4, wherein the fluid regulator is a capillary valve, a layer of mesh with pore size of 50 to 200 μm, or a thin film configured to rupture under an applied load.

6. The fluid separation device of claim 1, further comprising a support disposed at a lower portion of the separation chamber, the support being configured to support the membrane, wherein the support facilitates fluid communication between the membrane and the outlet of the sample collection receptacle.

7. The fluid separation device of claim 1, wherein the inlet is configured to contain a fixed volume of the fluid sample.

8. The fluid separation device of claim 7, wherein, when a force is applied to the device, the fluid distributor permits fluid communication between the inlet and the separation chamber by releasing the fixed volume of the fluid sample.

9. The fluid separation device of claim 1, wherein the at least one hydrophilic guide member extends from the outlet of the sample collection receptacle substantially vertically in a direction of the compression force.

10. The fluid separation device of claim 1, wherein the at least one hydrophilic guide member extends from the outlet of the sample collection receptacle at an angle of at least 10° from a horizontal plane perpendicular to a direction of the compression force.

11. The fluid separation device of claim 1, wherein the at least one hydrophilic guide member comprises a hydrophilic material, the hydrophilic material comprising a liquid drop contact angle θ<65°.

12. The fluid separation device of claim 1, wherein the inlet has a liquid drop contact angle θ<90°.

13. The fluid separation device of claim 1, wherein the membrane has an average porosity of no less than 40%.

14. The fluid separation device of claim 1, wherein the membrane at least has a minimum pore size of 0.1 to 2 μm.

15. A method for separating a fluid sample comprising:
applying a fluid sample to the fluid separation device of claim 1;
exerting a force onto the device to permit a volume of the fluid sample to pass through a fluid regulator into the separation chamber;
coupling the cap to the sample collection receptacle to exert a compression force;
separating the fluid sample in the membrane of the separation chamber; and
collecting a separated aliquot from the fluid sample via the outlet in fluid communication with the separation chamber.

16. The method of claim 15, wherein the fluid sample is a blood sample.

17. The method of claim 15, wherein the separated aliquot is plasma.

18. The method of claim 15, further comprising:
affixing a collection vessel to a flange of the outlet on the fluid separation device.

19. The method of claim 15, wherein the separated aliquot is collected in about 30-180 seconds.

20. A kit comprising:
one or more fluid separation devices of claim 1,
a means for obtaining a blood sample from a human or animal subject;
optionally, a blood collection vial;
optionally, one or more antiseptics;
optionally one or more pairs of gloves; and
packaging, labels, and instructions for use.
